(19) United States
(12) Patent Application Publication
(10) Pub. No.: US 2018/0273499 A1
(43) Pub. Date: Sep. 27, 2018

(54) SALTS AND SOLID STATE FORMS OF VORTIOXETINE

(71) Applicant: Teva Pharmaceuticals International GmbH, Jona (CH)

(72) Inventors: Sharona SHACHANTOV, Kfar-Saba (IL); Doron RUDIK, Modi'in (IL); Ariel MITTELMAN, Elad (IL); Rotem SELLA-EREZ, Tel-Aviv (IL); Yuval COHEN, Rishon LeZion (IL); Jonathan ENAV, Bnei-Brak (IL); Vera KESELMAN, Beer Sheva (IL); Gustavo FRENKEL, Beer Sheva (IL); Moshe BAR, Meitar (IL); Polina LAPIDO, Rishon LeZion (IL)

(73) Assignee: Teva Pharmaceuticals International GmbH, Jona (CH)

(21) Appl. No.: 15/924,510

(22) Filed: Mar. 19, 2018

(30) Foreign Application Priority Data
Mar. 17, 2017 (EP) 17.161585.9
Apr. 11, 2017 (EP) 17.166073.1
Jul. 12, 2017 (EP) 17.180965.0
Sep. 19, 2017 (EP) 17.191839.4
Sep. 26, 2017 (EP) 17.193320.3

(51) Int. Cl. C07D 295/096

(52) U.S. Cl. CPC C07D 295/096; C07B 2200/13

(57) ABSTRACT

The present disclosure relates to new salts of Vortioxetine, including butyrate, iso-butyrate, benzoate and propionate, solid state forms of the Vortioxetine salts, processes for the preparation thereof, pharmaceutical formulations/compositions thereof, and methods of use thereof.

### PRIORITY

This application claims priority to European Patent Application No. 17.161585.9 filed Mar. 17, 2017; European Patent Application No. 17.166073.1 filed Apr. 11, 2017; European Patent Application No. 17.180965.0 filed Jul. 12, 2017; European Patent Application No. 17.191839.4 filed Sep. 19, 2017; and European Patent Application No. 17.193320.3 filed Sep. 26, 2017; the contents of which are incorporated by reference herein in their entirety.

### TECHNICAL FIELD OF THE PRESENT INVENTION

The present disclosure relates to new salts of Vortioxetine, including butyrate, iso-butyrate, benzoate and propionate, and solid state forms of the Vortioxetine salts, processes for the preparation thereof, pharmaceutical formulations/compositions thereof, and methods of use thereof.

### BACKGROUND OF THE PRESENT INVENTION

Vortioxetine, 1-[2-[(2,4-dimethylphenyl)thio] phenyl]-, also called 1-[2-(2,4-Dimethylphenylsulfanyl)phenyl]piperazine, having the following formula:

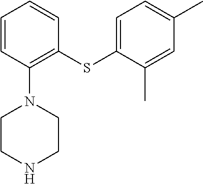 [SMILES: Cc1ccc(Sc2ccccc2N2CCNCC2)c(C)c1]

is a 5-HT3 and 5-HT7 receptor antagonist, 5-HT1B receptor partial agonist, 5-HT1A receptor agonist and inhibitor of the 5-HT transporter, which has been developed by Lundbeck for the treatment of major depressive disorder (MDD).

Vortioxetine was described in U.S. Pat. No. 7,144,884. Several crystalline forms of Vortioxetine hydrobromide, including forms alpha, beta and gamma and of Vortioxetine base are described in U.S. Pat. No. 8,722,684, U.S. Pat. No. 8,598,348, WO 2014/044721 and WO 2015/166379. Other salts and crystalline forms were also described for example in WO 2007/144005, U.S. Publication No. 2016/0289202, WO 2015/035802, WO 2015/114395 and WO 2016/180870. The crystalline forms exist in either solvate, anhydrous or hydrate forms.

Polymorphism, the occurrence of different crystal forms, is a property of some molecules and molecular complexes. A single compound, like Vortioxetine, may give rise to a variety of polymorphs having distinct crystal structures and physical properties like melting point, thermal behaviors (e.g. measured by thermogravimetric analysis—“TGA”, or differential scanning calorimetry—“DSC”), X-ray powder diffraction (XRPD) pattern, infrared absorption fingerprint, Raman absorption fingerprint, and solid state (¹³C—) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, improving the dissolution profile, or improving stability (polymorphic as well as chemical stability) and shelf-life. These variations in the properties of different salts and solid state forms may also provide improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to use variations in the properties and characteristics of a solid active pharmaceutical ingredient for providing an improved product.

Discovering new salts and solid state forms and solvates of a pharmaceutical product can provide materials having desirable processing properties, such as ease of handling, ease of processing, storage stability, and ease of purification or as desirable intermediate crystal forms that facilitate conversion to other salts or polymorphic forms. New polymorphic forms and solvates of a pharmaceutically useful compound can also provide an opportunity to improve the performance characteristics of a pharmaceutical product (dissolution profile, bioavailability, etc.). It enlarges the repertoire of materials that a formulation scientist has available for formulation optimization, for example by providing a product with different properties, e.g., a different crystal habit, higher crystallinity or polymorphic stability which may offer better processing or handling characteristics, improved dissolution profile, or improved shelf-life. For at least these reasons, there is a need for additional salts and solid state forms (including solvated forms) of Vortioxetine.

### SUMMARY OF THE PRESENT INVENTION

The present disclosure relates to new salts of Vortioxetine, including butyrate, iso-butyrate, benzoate and propionate, and solid state forms of the Vortioxetine salts.

The present disclosure also relates to the uses of the salts and solid state forms of Vortioxetine salts of the present disclosure, for preparing other salts of Vortioxetine and solid state forms of Vortioxetine and Vortioxetine salts.

The present disclosure also encompasses the uses of the above described salts and solid state forms of Vortioxetine salts for the preparation of pharmaceutical compositions and/or formulations.

In another embodiment, the present disclosure encompasses pharmaceutical compositions comprising the above described salts and solid state forms of Vortioxetine salts.

In yet another embodiment, the present disclosure encompasses pharmaceutical formulations comprising the above described salts and solid state forms of Vortioxetine salts, and at least one pharmaceutically acceptable excipient.

The present disclosure further encompasses processes to prepare said pharmaceutical formulations of Vortioxetine comprising combining any one of the above salts and solid state forms of Vortioxetine salts, or pharmaceutical compositions comprising them, and at least one pharmaceutically acceptable excipient.

The salts and solid state forms of the Vortioxetine salts defined herein as well as the pharmaceutical compositions and formulations of the salts and solid state form of the Vortioxetine salts can be used as medicaments, particularly for the treatment of major depressive disorder (MDD), comprising administering a therapeutically effective amount of any of the salts and solid state form of the salts of the present disclosure, or at least one of the above pharmaceutical compositions or formulations, to a subject suffering from major depressive disorder (MDD), or otherwise in need of the treatment.

The present disclosure also provides the uses of the salts and solid state forms of Vortioxetine of the present disclosure, or at least one of the above pharmaceutical compositions or formulations, for the manufacture of medicaments for treating major depressive disorder (MDD).

In another aspect, the present disclosure encompasses the above salts and solid state forms of Vortioxetine salts for use in medicine, preferably for the treatment of major depressive disorder (MDD).

### BRIEF DESCRIPTION OF THE DRAWINGS

FIG. 1 shows an X-ray powder diffraction (XRPD) pattern of Form B1 of Vortioxetine butyrate

FIG. 2 shows an XRPD pattern of Form B2 of Vortioxetine butyrate

FIG. 3 shows an XRPD pattern of Form BZ1 of Vortioxetine benzoate

FIG. 4 shows an XRPD pattern of Form BZ2 of Vortioxetine benzoate

FIG. 5 shows an XRPD pattern of Form BZ3 of Vortioxetine benzoate

FIG. 6 shows an XRPD pattern of Form IB2 of Vortioxetine iso butyrate

FIG. 7 shows an XRPD pattern of Form IB4 of Vortioxetine iso butyrate

FIG. 8 shows an XRPD pattern of Form P1 of Vortioxetine propionate

FIG. 9 shows an XRPD pattern of Form P3 of Vortioxetine propionate

FIG. 10 shows an XRPD pattern of Form P4 of Vortioxetine propionate

FIG. 11 shows an XRPD pattern of Form IB5 of Vortioxetine iso butyrate

FIG. 12 shows an XRPD pattern of Form BZ4 of Vortioxetine benzoate

FIG. 13 shows an XRPD pattern of Form BZ5 of Vortioxetine benzoate

FIG. 14 shows an XRPD pattern of Form BZ6 of Vortioxetine benzoate

FIG. 15 shows an XRPD pattern of Form BZ7 of Vortioxetine benzoate

FIG. 16 shows an XRPD pattern of BZ8 of Vortioxetine benzoate

FIG. 17 shows an XRPD pattern of a crystalline Vortioxetine base, as described in U.S. Pat. No. 8,722,684.

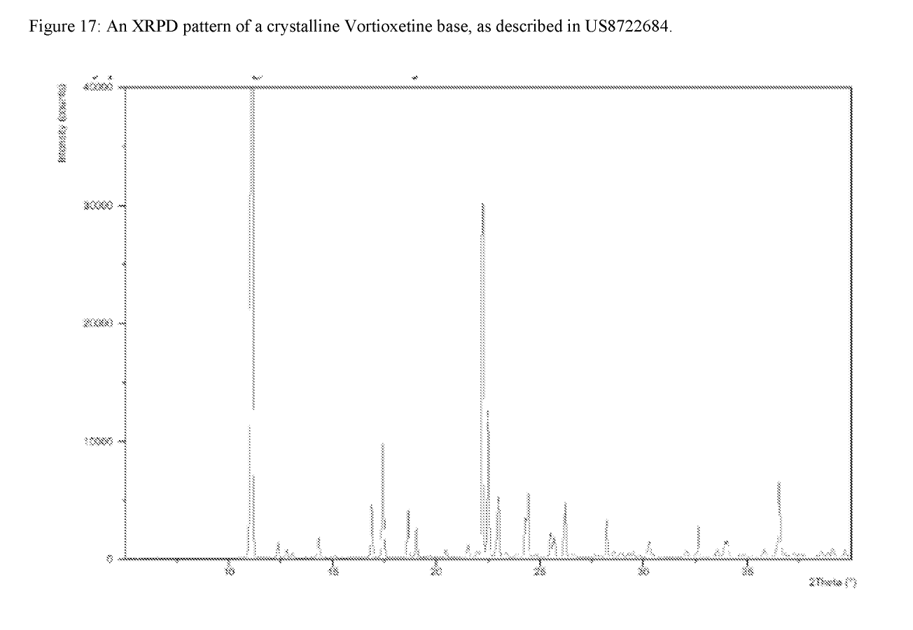

### DETAILED DESCRIPTION OF ILLUSTRATIVE EMBODIMENTS

The present disclosure relates to salts and solid state forms of Vortioxetine salts, to processes for preparation thereof and to pharmaceutical compositions and formulations comprising these salts and solid state forms of the salts and/or combinations thereof. The disclosure also relates to the conversion of the Vortioxetine salts and solid state forms of the Vortioxetine salts of the present disclosure to other salts and/or solid state form of Vortioxetine and Vortioxetine salts.

The Vortioxetine salts and solid state forms of Vortioxetine salts according to the present disclosure may have advantageous properties selected from at least one of: chemical or polymorphic purity, flowability, solubility, dissolution rate, bioavailability, morphology or crystal habit, stability—such as chemical stability as well as thermal and mechanical stability with respect to polymorphic conversion, stability towards dehydration and/or storage stability, a lower degree of hygroscopicity, low content of residual solvents, adhesive tendencies and advantageous processing and handling characteristics such as compressibility, and bulk density.

A crystal form may be referred to herein as being characterized by graphical data “as depicted in” a Figure. Such data include, for example, powder X-ray diffractograms and solid state NMR spectra. As is well-known in the art, the graphical data potentially provides additional technical information to further define the respective solid state form (a so-called “fingerprint”) which can not necessarily be described by reference to numerical values or peak positions alone. In any event, the skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms.

A Vortioxetine salt or crystal form of a Vortioxetine salt referred to herein as being characterized by graphical data “as depicted in” a Figure will thus be understood to include any Vortioxetine salt or crystal form of the Vortioxetine salt, characterized with the graphical data having such small variations, as are well known to the skilled person, in comparison with the Figure.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, solid states of Vortioxetine salts described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject solid state form of Vortioxetine salt. In some embodiments of the disclosure, the described salts and solid state forms of Vortioxetine salts may contain from about 1% to about 20% (w/w), from about 5% to about 20% (w/w), or from about 5% to about 10% (w/w) of one or more other salts or solid state forms of the same Vortioxetine salt.

As used herein, unless stated otherwise, XRPD peaks reported herein are preferably measured using CuKα radiation, λ=1.5418 Å, preferably, XRPD peaks reported herein are measured using CuK α radiation, λ=1.5418 Å, at a temperature of 25±3° C.

As used herein, the term “isolated” in reference to salts and solid state forms of Vortioxetine salts of the present disclosure corresponds to salt and/or solid state form of Vortioxetine salts that is physically separated from the reaction mixture in which it is formed.

A thing, e.g., a reaction mixture, may be characterized herein as being at, or allowed to come to “room temperature”, often abbreviated “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

A process or step may be referred to herein as being carried out “overnight.” This refers to a time interval, e.g., for the process or step, that spans the time during the night, when that process or step may not be actively observed. This time interval is from about 8 to about 20 hours, or about 10 to about 18 hours, typically about 16 hours.

As used herein, the expression “wet crystalline form” refers to a polymorph that was not dried using any conventional techniques to remove residual solvent. Examples for such conventional techniques can be, but not limited to, evaporation, vacuum drying, oven drying, drying under nitrogen flow, and the like.

As used herein, the expression “dry crystalline form” refers to a polymorph that was dried using any conventional techniques to remove residual solvent. Examples of such conventional techniques can be, but are not limited to, evaporation, vacuum drying, oven drying, drying under nitrogen flow, and the like.

As used herein, and unless stated otherwise, the term “anhydrous” in relation to a Vortioxetine salt or a crystalline Vortioxetine salt relates to a Vortioxetine salt or crystalline Vortioxetine salt which does not include any crystalline water (or other solvents) in a defined, stoichiometric amount within the crystal. Moreover, an “anhydrous” form does not contain more than about 1% (w/w) of either water or organic solvents as measured for example by TGA.

The term “solvate”, as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

The amount of solvent employed in a chemical process, e.g., a reaction or a crystallization may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding (methyl tert-butyl ether) MTBE (1.5 v/v) to a 100 ml reaction mixture would indicate that 150 mL of MTBE was added.

As used herein the term non-hygroscopic in relation to a Vortioxetine salt or crystalline form thereof, refers to less than about 1.0% (w/w) absorption of water at about 25° C. and about 80% relative humidity (RH), by the Vortioxetine salt or crystalline form thereof as determined for example by TGA. Water can be for example atmospheric water.

As used herein, the term “reduced pressure” refers to a pressure of about 10 mbar to about 500 mbar.

As used herein, and unless indicated otherwise, the term “thermo-dynamical stability” in relation to salts and solid state forms of Vortioxetine salts refers to resistance of the solid state form to polymorphic conversion under certain conditions, for example, heating, melting or dissolving. In some embodiments, the term refers to less than about 20% (w/w), about 10% (w/w), about 5% (w/w), about 1% (w/w), about 0.5% (w/w), or about 0% (w/w) conversion of the crystalline Vortioxetine salt to any other solid state form of Vortioxetine or a salt thereof as measured by XRPD. In some embodiments, the conversion is about 1% (w/w) to about 20% (w/w), about 1% (w/w) to about 10% (w/w) or about 1% (w/w) to about 5% (w/w).

The present disclosure comprises Vortioxetine salts selected from: Vortioxetine butyrate, Vortioxetine iso-butyrate, Vortioxetine benzoate and Vortioxetine propionate as well as solid state forms thereof.

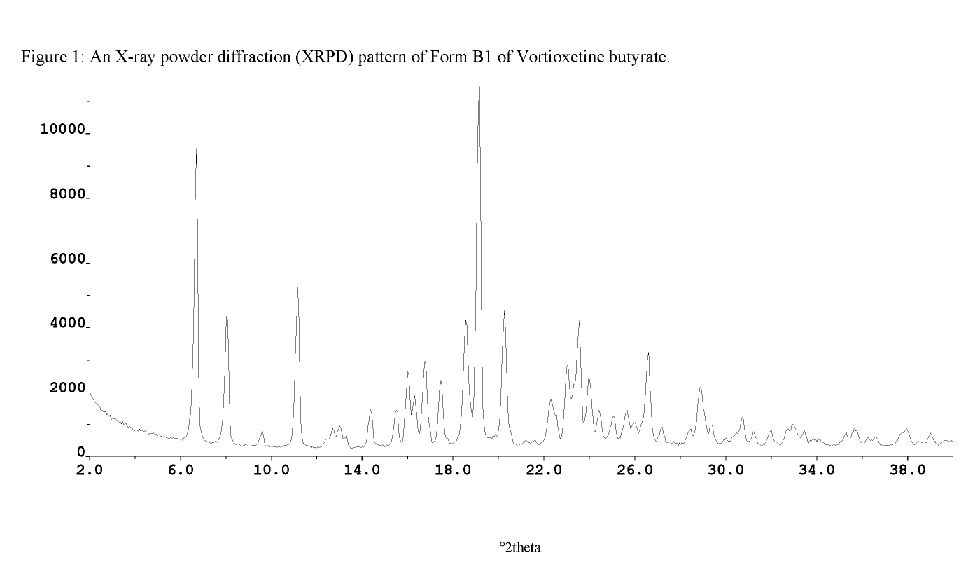

The present disclosure comprises a crystalline form of Vortioxetine butyrate designated as Form B1. The crystalline Form B1 of Vortioxetine butyrate can be characterized by data selected from one or more of the following: an XRPD pattern having peaks at 6.7, 9.6, 15.5, 16.0 and 27.2 degrees 2-theta±0.2 degrees 2-theta; an XRPD pattern substantially as depicted in FIG. 1; or combinations of these data.

Crystalline Form B1 of Vortioxetine butyrate may be further characterized by data selected from one or more of the following: an XRPD pattern having peaks at 6.7, 9.6, 15.5, 16.0 and 27.2 degrees two theta±0.2 degrees two theta; and also having one, two, three, four or five additional peaks selected from 16.8, 20.2, 23.5, 24.0 and 26.6 degrees two theta±0.2 degrees two theta; or combinations of these data.

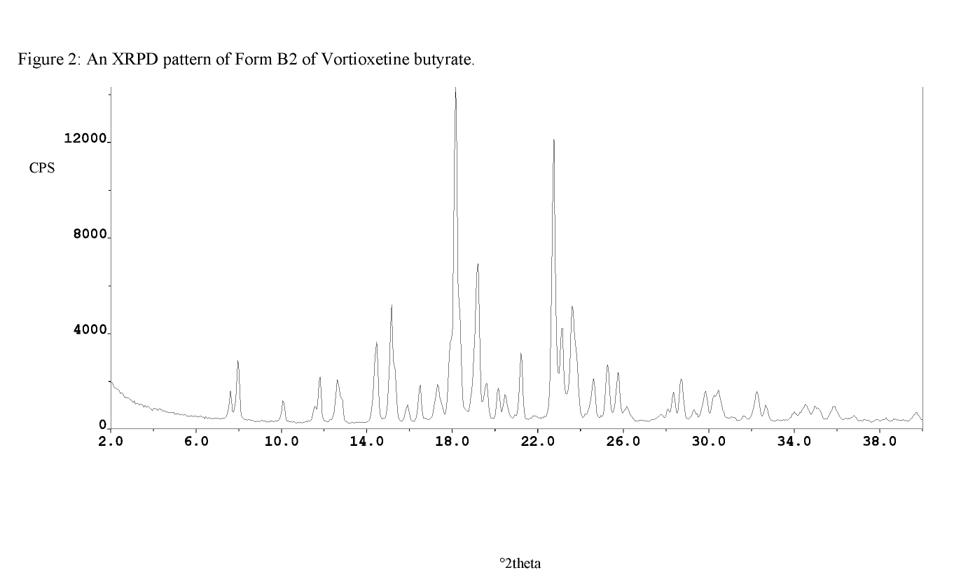

The present disclosure further comprises a crystalline form of Vortioxetine butyrate designated as Form B2. The crystalline Form B2 of Vortioxetine butyrate can be characterized by data selected from one or more of the following: an XRPD pattern having peaks at 7.6, 10.1, 11.8, 15.2 and 18.1 degrees two theta±0.2 degrees two theta; an XRPD pattern substantially as depicted in FIG. 2; or combinations of these data.

Crystalline Form B2 of Vortioxetine butyrate may be further characterized by data selected from one or more of the following: an XRPD pattern having peaks at 7.6, 10.1, 11.8, 15.2 and 18.1 degrees two theta±0.2 degrees two theta; and also having one, two, three, four or five additional peaks selected from 16.5, 21.2, 22.8, 25.3 and 29.8 degrees two theta±0.2 degrees two theta; or combinations of these data.

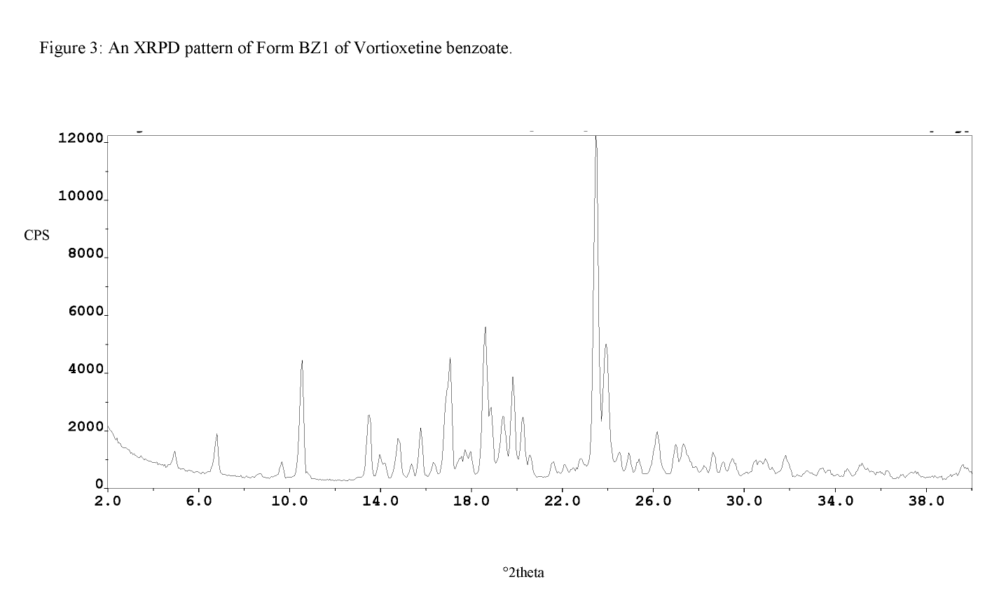

The present disclosure also comprises a crystalline form of Vortioxetine benzoate designated as Form BZ1. The crystalline Form BZ1 of Vortioxetine benzoate can be characterized by data selected from one or more of the following: an XRPD pattern having peaks at 6.7, 9.5, 10.4, 14.7 and 16.9 degrees two theta±0.2 degrees two theta; an XRPD pattern substantially as depicted in FIG. 3; or combinations of these data.

Crystalline Form BZ1 of Vortioxetine benzoate may be further characterized by data selected from one or more of the following: an XRPD pattern having peaks at 6.7, 9.5, 10.4, 14.7 and 16.9 degrees two theta±0.2 degrees two theta; and also having one, two, three, four or five additional peaks selected from 4.8, 18.7, 19.3, 23.4 and 23.8 degrees two theta±0.2 degrees two theta; or combinations of these data.

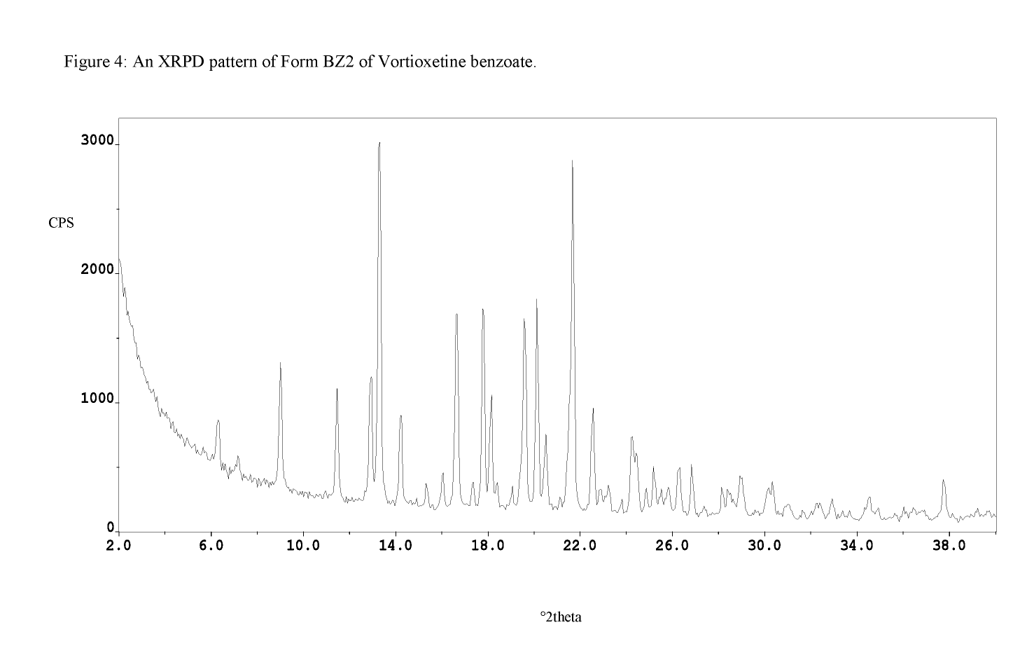

The present disclosure also comprises a crystalline form of Vortioxetine benzoate designated as Form BZ2. The crystalline Form BZ2 of Vortioxetine benzoate can be characterized by data selected from one or more of the following: an XRPD pattern having peaks at 6.3, 7.1, 9.0, 11.5 and 21.7 degrees two theta±0.2 degrees two theta; an XRPD pattern substantially as depicted in FIG. 4; or combinations of these data.

Crystalline Form BZ2 of Vortioxetine benzoate may be further characterized by data selected from one or more of the following: an XRPD pattern having peaks at 6.3, 7.1, 9.0, 11.5 and 21.7 degrees two theta±0.2 degrees two theta; and also having one, two, three, four or five additional peaks selected from 13.3, 14.2, 16.7, 18.1 and 22.6 degrees two theta±0.2 degrees two theta; or combinations of these data.

Typically, form BZ2 of Vortioxetine benzoate may be an anhydrous form.

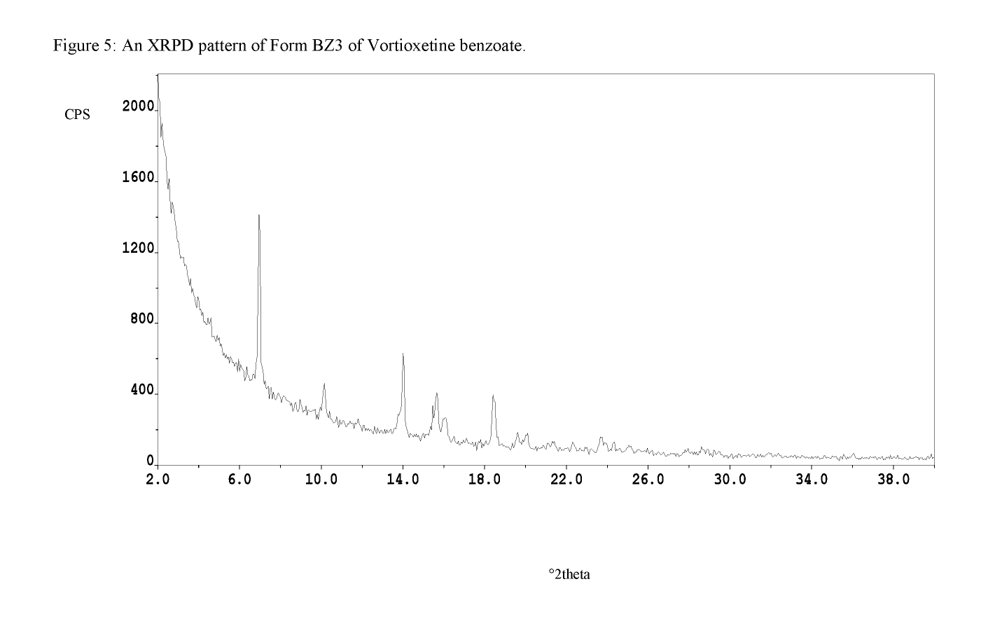

The present disclosure further comprises a crystalline form of Vortioxetine benzoate designated as Form BZ3. The crystalline Form BZ3 of Vortioxetine benzoate can be characterized by data selected from one or more of the following: an XRPD pattern having peaks at 7.0, 10.1, 14.0, 15.7 and 18.5 degrees two theta±0.2 degrees two theta; an XRPD pattern substantially as depicted in FIG. 5; or combinations of these data.

Crystalline Form BZ3 of Vortioxetine benzoate may be further characterized by data selected from one or more of the following: an XRPD pattern having peaks at 7.0, 10.1, 14.0, 15.7 and 18.5 degrees two theta±0.2 degrees two theta; and also having one, two, three, four or five additional peaks selected from 16.0, 19.6, 20.0, 23.7 and 24.3 degrees two theta±0.2 degrees two theta; or combinations of these data.

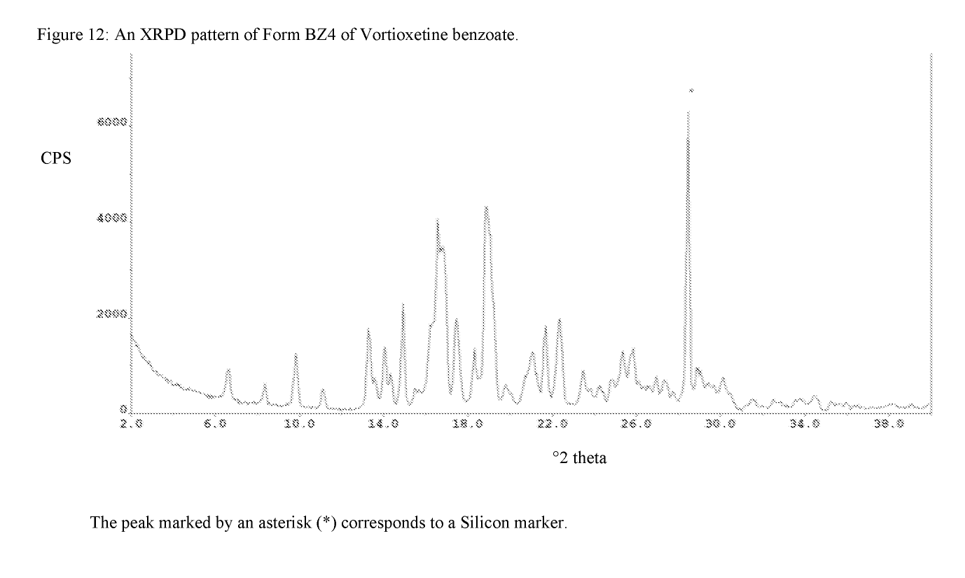

The present disclosure further comprises a crystalline form of Vortioxetine benzoate designated as Form BZ4. The crystalline Form BZ4 of Vortioxetine benzoate can be characterized by data selected from one or more of the following: an XRPD pattern having peaks at 8.4, 9.8, 14.9, 18.9 and 22.4 degrees two theta±0.2 degrees two theta; an XRPD pattern substantially as depicted in FIG. 12; or combinations of these data.

Crystalline Form BZ4 of Vortioxetine benzoate may be further characterized by data selected from one or more of the following: an XRPD pattern having peaks at 8.4, 9.8, 14.9, 18.9 and 22.4 degrees two theta±0.2 degrees two theta; and also having one, two, three, four or five additional peaks selected from 11.1, 13.3, 16.6, 17.5 and 21.1 degrees two theta±0.2 degrees two theta; or combinations of these data.

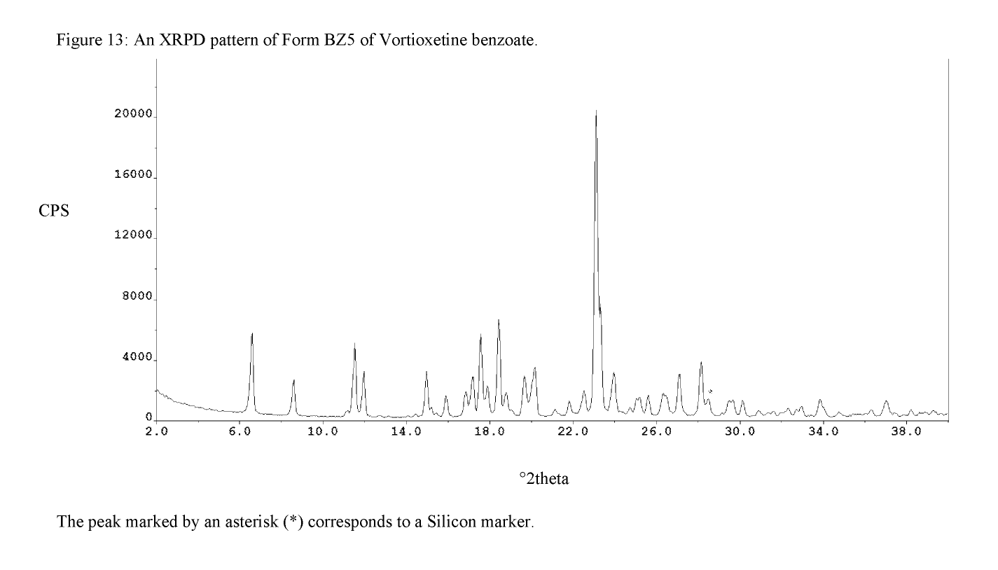

The present disclosure further comprises a crystalline form of Vortioxetine benzoate designated as Form BZ5. The crystalline Form BZ5 of Vortioxetine benzoate can be characterized by data selected from one or more of the following: an XRPD pattern having peaks at 6.6, 8.6, 11.5, 12.0 and 23.1 degrees two theta±0.2 degrees two theta; an XRPD pattern substantially as depicted in FIG. 13; or combinations of these data.

Crystalline Form BZ5 of Vortioxetine benzoate may be further characterized by data selected from one or more of the following: an XRPD pattern having peaks at 6.6, 8.6, 11.5, 12.0 and 23.1 degrees two theta±0.2 degrees two theta; and also having one, two, three, four or five additional peaks selected from 15.0, 17.2, 17.6, 18.4, 28.1 degrees two theta±0.2 degrees two theta; or combinations of these data.

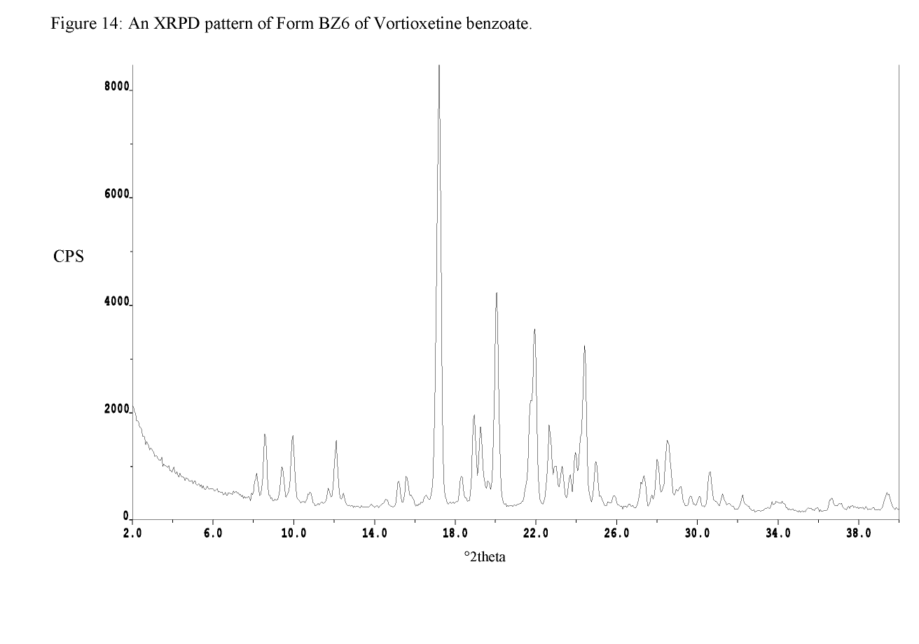

The present disclosure further comprises a crystalline form of Vortioxetine benzoate designated as Form BZ6. The crystalline Form BZ6 of Vortioxetine benzoate can be characterized by data selected from one or more of the following: an XRPD pattern having peaks at 8.1, 8.5, 17.2, 19.3, and 20.0 degrees two theta±0.2 degrees two theta; an XRPD pattern substantially as depicted in FIG. 14; or combinations of these data.

Crystalline Form BZ6 of Vortioxetine benzoate may be further characterized by data selected from one or more of the following: an XRPD pattern having peaks at 8.1, 8.5, 17.2, 19.3, and 20.0 degrees two theta±0.2 degrees two theta; and also having one, two, three, four or five additional peaks selected from 9.4, 12.1, 15.2, 22.0 and 24.4 degrees two theta±0.2 degrees two theta; or combinations of these data.

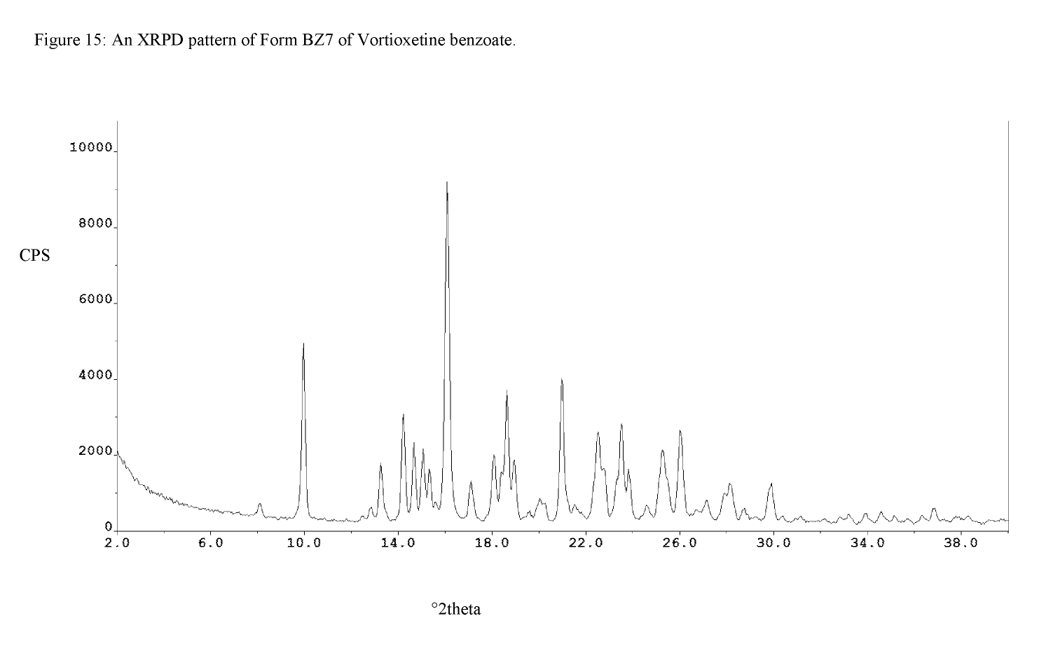

The present disclosure further comprises a crystalline form of Vortioxetine benzoate designated as Form BZ7. The crystalline Form BZ7 of Vortioxetine benzoate can be characterized by data selected from one or more of the following: an XRPD pattern having peaks at 9.9, 13.2, 16.0, 20.9 and 26.0 degrees two theta±0.2 degrees two theta; an XRPD pattern substantially as depicted in FIG. 15; or combinations of these data.

Crystalline Form BZ7 of Vortioxetine benzoate may be further characterized by data selected from one or more of the following: an XRPD pattern having peaks at 9.9, 13.2, 16.0, 20.9 and 26.0 degrees two theta±0.2 degrees two theta; and also having one, two, three, four or five additional peaks selected from 14.2, 14.7, 18.6, 22.5 and 23.5 degrees two theta±0.2 degrees two theta; or combinations of these data.

Typically, form BZ7 of Vortioxetine benzoate may be an anhydrous form.

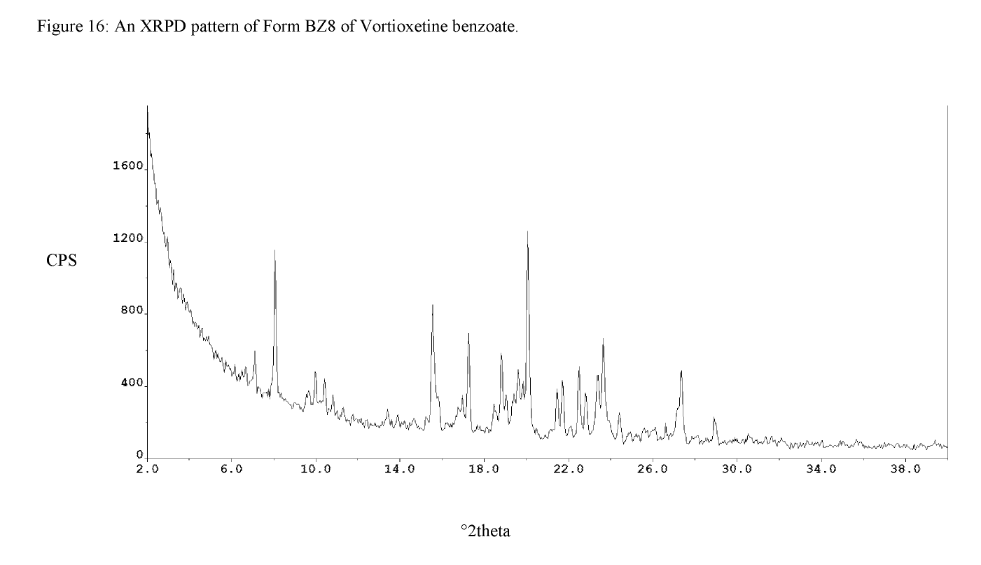

The present disclosure further comprises a crystalline form of Vortioxetine benzoate designated as Form BZ8. The crystalline Form BZ8 of Vortioxetine benzoate can be characterized by data selected from one or more of the following: an XRPD pattern having peaks at 7.1, 8.1, 15.6, 17.3 and 20.1 degrees two theta±0.2 degrees two theta; an XRPD pattern substantially as depicted in FIG. 16; or combinations of these data.

Crystalline Form BZ8 of Vortioxetine benzoate may be further characterized by data selected from one or more of the following: an XRPD pattern having peaks at 7.1, 8.1, 15.6, 17.3 and 20.1 degrees two theta±0.2 degrees two theta; and also having one, two, three, four or five additional peaks selected from 10, 10.4, 18.8, 23.6 and 27.4 degrees two theta±0.2 degrees two theta; or combinations of these data.

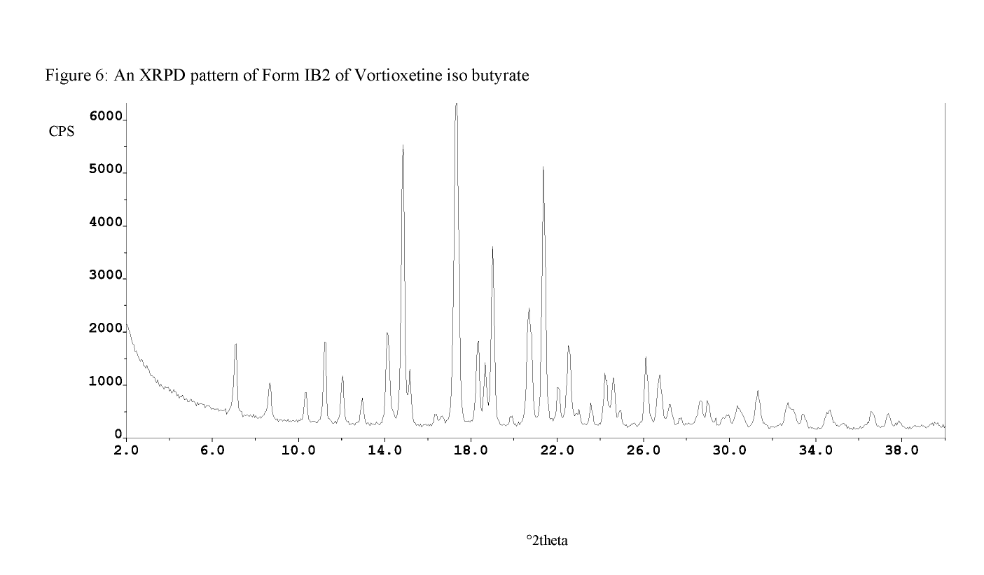

The present disclosure further comprises a crystalline form of Vortioxetine iso butyrate designated as Form IB2. The crystalline Form IB2 of Vortioxetine iso butyrate can be characterized by data selected from one or more of the following: an XRPD pattern having peaks at 7.1, 8.7, 10.3, 12.0, and 14.8 degrees two theta±0.2 degrees two theta; an XRPD pattern substantially as depicted in FIG. 6; or combinations of these data.

Crystalline Form IB2 of Vortioxetine iso butyrate may be further characterized by data selected from one or more of the following: an XRPD pattern having peaks at 7.1, 8.7, 10.3, 12.0 and 14.8 degrees two theta±0.2 degrees two theta; and also having one, two, three, four or five additional peaks selected from 14.1, 17.3, 19.0, 20.7 and 21.4 degrees two theta±0.2 degrees two theta; or combinations of these data.

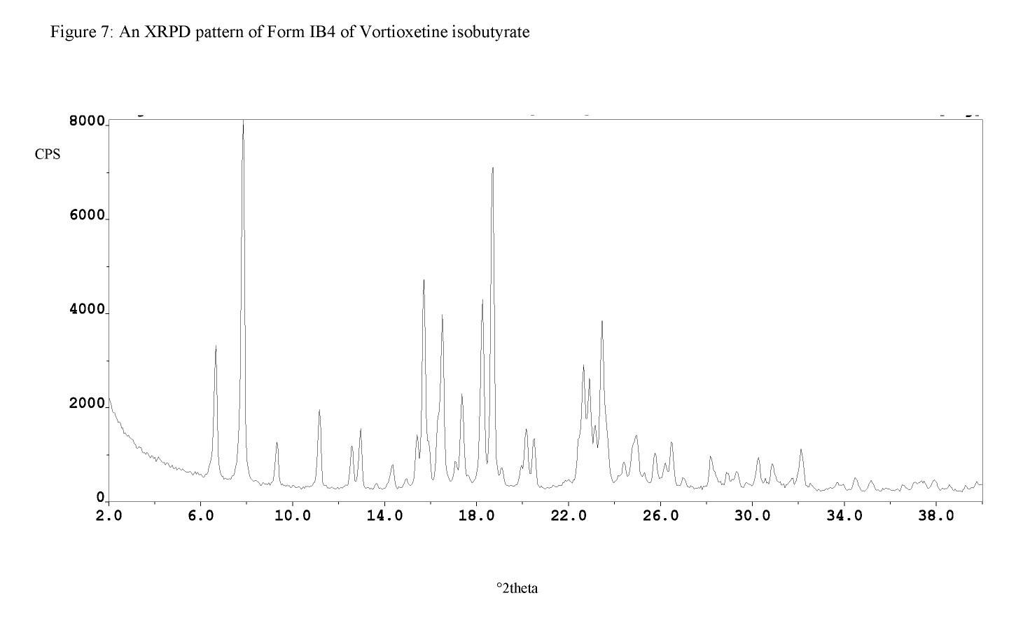

The present disclosure further comprises a crystalline form of Vortioxetine iso butyrate designated as Form IB4. The crystalline Form IB4 of Vortioxetine iso butyrate can be characterized by data selected from one or more of the following: an XRPD pattern having peaks at 6.6, 15.7, 18.7, 20.2 and 23.5 degrees two theta±0.2 degrees two theta; an XRPD pattern substantially as depicted in FIG. 7; or combinations of these data.

Crystalline Form IB4 of Vortioxetine iso butyrate may be further characterized by data selected from one or more of the following: an XRPD pattern having peaks at 6.6, 15.7, 18.7, 20.2 and 23.5 degrees two theta±0.2 degrees two theta; and also having one, two, three, four or five additional peaks selected from 7.8, 12.6, 12.9, 18.2 and 20.5 degrees two theta±0.2 degrees two theta; or combinations of these data.

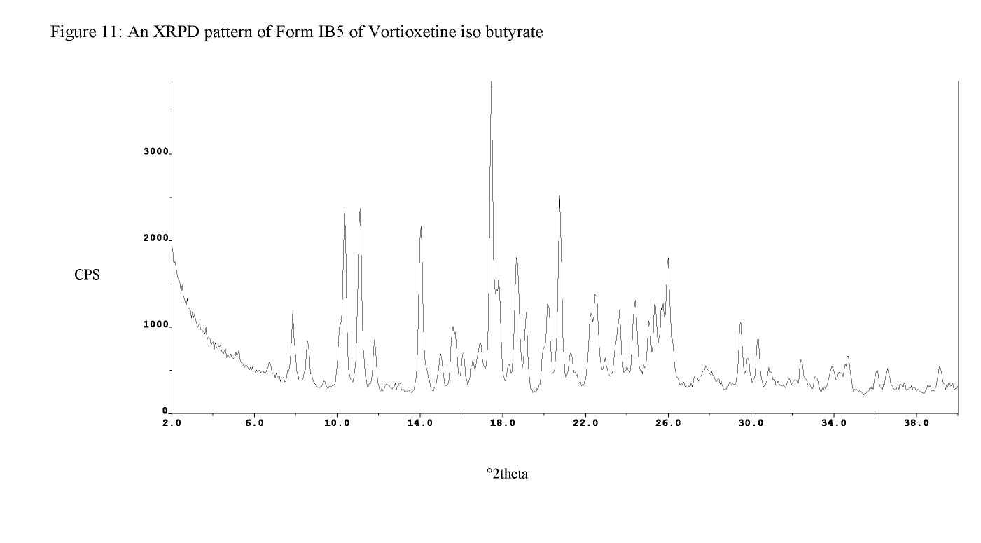

The present disclosure further comprises a crystalline form of Vortioxetine iso butyrate designated as Form IB5. The crystalline Form IB5 of Vortioxetine iso butyrate can be characterized by data selected from one or more of the following: an XRPD pattern having peaks at 10.4, 11.8, 14.0, 20.2 and 26.0 degrees two theta±0.2 degrees two theta; an XRPD pattern substantially as depicted in FIG. 11; or combinations of these data.

Crystalline Form IB5 of Vortioxetine iso butyrate may be further characterized by data selected from one or more of the following: an XRPD pattern having peaks at 10.4, 11.8, 14.0, 20.2 and 26.0 degrees two theta±0.2 degrees two theta; and also having one, two, three, four or five additional peaks selected from 8.6, 16.1, 17.8, 25.1 and 25.4 degrees two theta±0.2 degrees two theta; or combinations of these data.

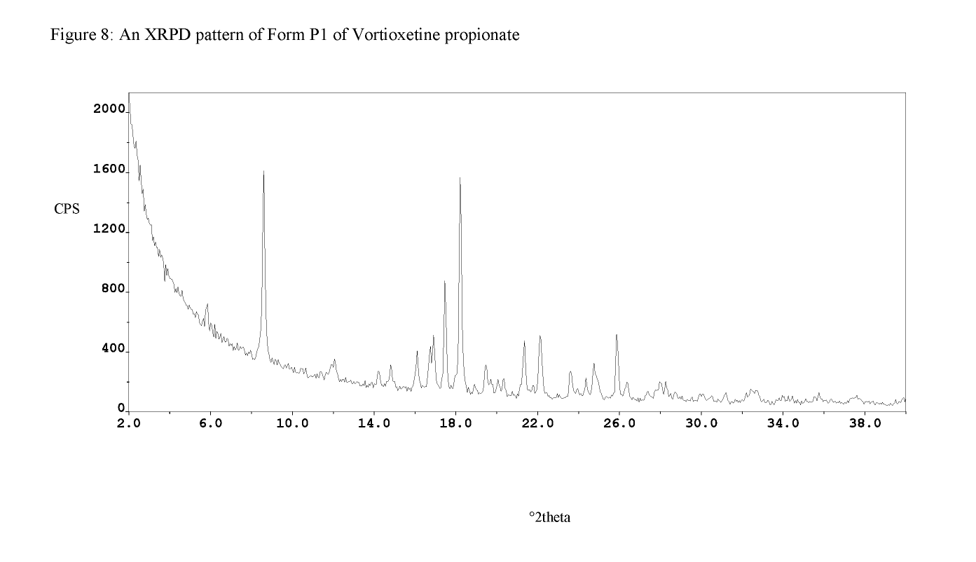

The present disclosure also comprises a crystalline form of Vortioxetine propionate designated as Form P1. The crystalline Form P1 of Vortioxetine propionate can be characterized by data selected from one or more of the following: an XRPD pattern having peaks at 8.6, 16.1, 18.2, 21.3 and 22.1 degrees two theta±0.2 degrees two theta; an XRPD pattern substantially as depicted in FIG. 8; or combinations of these data.

Crystalline Form P1 of Vortioxetine propionate may be further characterized by data selected from one or more of the following: an XRPD pattern having peaks at 8.6, 16.1, 18.2, 21.3 and 22.1 degrees two theta±0.2 degrees two theta; and also having one, two, three, four or five additional peaks selected from 16.8, 17.5, 19.5, 23.7 and 25.9 degrees two theta±0.2 degrees two theta; or combinations of these data.

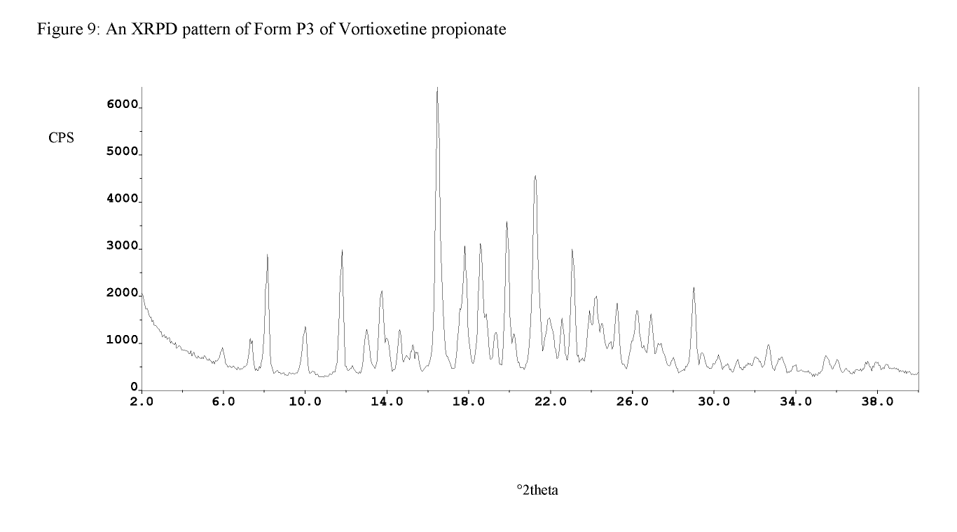

The present disclosure further comprises a crystalline form of Vortioxetine propionate designated as Form P3. The crystalline Form P3 of Vortioxetine propionate can be characterized by data selected from one or more of the following: an XRPD pattern having peaks at 7.2, 8.0, 9.8, 11.6 and 13.6 degrees two theta±0.2 degrees two theta; an XRPD pattern substantially as depicted in FIG. 9; or combinations of these data.

Crystalline Form P3 of Vortioxetine propionate may be further characterized by data selected from one or more of the following: an XRPD pattern having peaks at 7.2, 8.0, 9.8, 11.6 and 13.6 degrees two theta±0.2 degrees two theta; and also having one, two, three, four or five additional peaks selected from 16.3, 17.7, 18.4, 19.7 and 21.1 degrees two theta±0.2 degrees two theta; or combinations of these data.

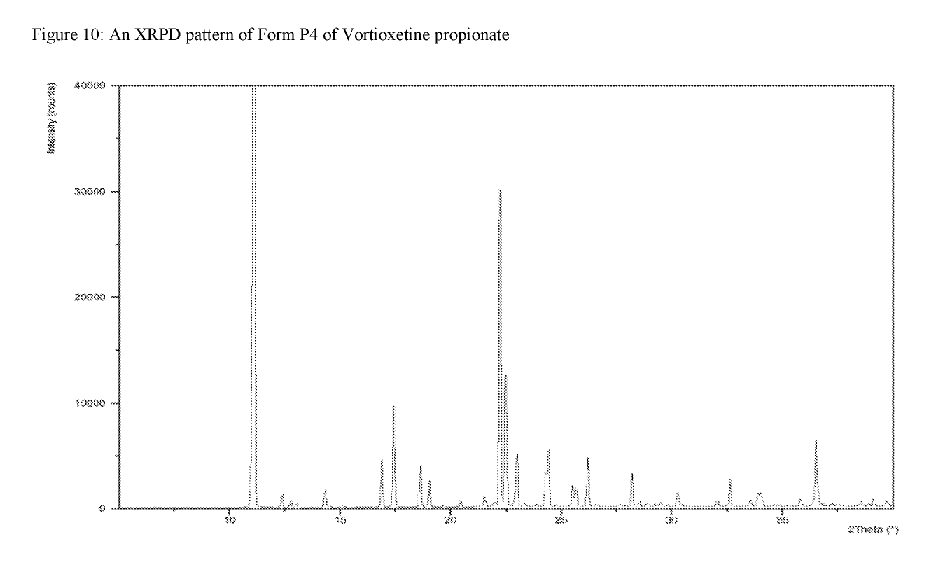

The present disclosure further comprises a crystalline form of Vortioxetine propionate designated as Form P4. The crystalline Form P4 of Vortioxetine propionate can be characterized by data selected from one or more of the following: an XRPD pattern having peaks at 6.6, 11.1, 20.6, 29.7 and 33.7 degrees two theta±0.2 degrees two theta; an XRPD pattern substantially as depicted in FIG. 10; or combinations of these data.

Crystalline Form P4 of Vortioxetine propionate may be further characterized by data selected from one or more of the following: an XRPD pattern having peaks at 6.6, 11.1, 20.6, 29.7 and 33.7 degrees two theta±0.2 degrees two theta; and also having one, two, three, four or five additional peaks selected from 8.1, 16.2, 18.9, 19.7 and 27.1 degrees two theta±0.2 degrees two theta; or combinations of these data.

The present disclosure also provides the use of the salts and solid state forms of Vortioxetine salts of the present disclosure for preparing other salts, solid state forms of Vortioxetine and/or solid state forms of Vortioxetine salts.

In another embodiment the present disclosure encompasses the use of any one of the above described salts or solid state form of Vortioxetine salts, or combinations thereof, for the preparation of pharmaceutical compositions and/or formulations.

The present disclosure further provides pharmaceutical compositions comprising any one of the above described salts or solid state forms of Vortioxetine salts, or combinations thereof.

In yet another embodiment, the present disclosure encompasses pharmaceutical formulations comprising any one of the above described salts or solid state forms of Vortioxetine salts, and/or combinations thereof, and at least one pharmaceutically acceptable excipient.

The present disclosure moreover encompasses processes to prepare said formulations of Vortioxetine salts and solid state forms thereof comprising combining any one of the above salts or solid state forms of Vortioxetine salts, and/or combinations thereof, and at least one pharmaceutically acceptable excipient.

In another embodiment, the present disclosure encompasses any one of the above described salts and solid state forms of Vortioxetine salts, or combinations thereof, for use in medicine, preferably for the treatment of major depressive disorder (MDD).

The present disclosure also provides methods of treating major depressive disorder (MDD), comprising administering a therapeutically effective amount of any one of the salts and solid state forms of Vortioxetine salts of the present disclosure or combinations thereof, or at least one of the above pharmaceutical compositions or formulations, to a subject suffering from major depressive disorder (MDD), or otherwise in need of the treatment.

The present disclosure also provides the use of any one of the Vortioxetine salts or solid state forms of Vortioxetine salts of the present disclosure, or combinations thereof, or at least one of the above pharmaceutical compositions or formulations, for the manufacture of a medicament for treating major depressive disorder (MDD).

The present disclosure further provides the Vortioxetine salts and solid state forms of Vortioxetine salts of the present disclosure, or combinations thereof, or at least one of the above pharmaceutical compositions or formulations for use in medicine, especially for treating major depressive disorder (MDD).

Having described the disclosure with reference to certain preferred embodiments, other embodiments will become apparent to one skilled in the art from consideration of the specification. The disclosure is further illustrated by reference to the following examples describing in detail the preparation of the composition and methods of use of the disclosure. It will be apparent to those skilled in the art that many modifications, both to materials and methods, may be practiced without departing from the scope of the disclosure.

Analytical Methods:

X-ray Powder diffraction pattern (“XRPD”) method:

Powder X-ray Diffraction was performed on ARL (SCINTAG) powder X-Ray diffractometer model X'TRA equipped with a solid state detector. Copper radiation of 1.5418 Å was used. Scanning parameters: range: 2-40 degrees two-theta; scan mode: continuous scan; step size: 0.05°, and a rate of 2 deg/min.

Vortioxetine base, characterized by XRPD substantially as depicted in FIG. 16, which is used as the starting material, may be prepared for example according to the processes described in U.S. Pat. No. 8,722,684 referenced above.

### EXAMPLES

### Example 1: Preparation of Form B1 of Vortioxetine Butyrate

To a mixture of Vortioxetine free base (1 g, 1 eq) and water (10 vol) butyric acid (1.1 eq) was added. The slurry mixture was stirred for 1 day at RT, the solid was filtered to provide Form B1 of Vortioxetine butyrate, as characterized by X-ray powder diffractogram (FIG. 1).

### Example 2: Preparation of Form B2 of Vortioxetine Butyrate

To a mixture of Vortioxetine free base (1 g, 1 eq) and ethyl acetate (10 vol) butyric acid (1.1 eq) was added. The slurry mixture was stirred for 1 day at RT, the solid was filtered to provide Form B2 of Vortioxetine butyrate, as characterized by X-ray powder diffractogram (FIG. 2).

### Example 3: Preparation of Form B2 of Vortioxetine Butyrate

Vortioxetine free base (10 g, 1 eq) and ethyl acetate (5 vol) were charged in 250 ml reactor. The stirred mixture was heated to 78° C. and butyric acid (1.1 eq) was added. The obtained clear solution was cooled to 25° C. during 1.5 hr. A light slurry mixture was obtained and was further cooled to 0° C. and stirred for 2 hr. The solid was filtered and dried in vacuum oven at 40° C. overnight to provide Form B2 of Vortioxetine butyrate, as was confirmed by XRPD.

### Example 4: Preparation of Form BZ1 of Vortioxetine Benzoate

To a mixture of Vortioxetine free base (15 g, 1 eq) and MIBK (150 ml, 10 vol) benzoic acid (4.82 g, 1.1 eq) was added. The slurry mixture was stirred for 80 hr. at RT, the solid was filtered and dried in vacuum oven at 40° C. overnight to provide Vortioxetine benzoate Form BZ1, as was characterized by X-ray powder diffractogram (FIG. 3).

### Example 5: Preparation of Form BZ2 of Vortioxetine Benzoate

Vortioxetine free base (15 g, 1 eq) and ethyl acetate (150 ml, 10 vol) were charged in 250 ml reactor and benzoic acid (4.82 g, 1.1 eq) was added. The mixture was stirred for 80 hr. and the solid was filtered and dried in vacuum oven at 40° C. overnight. The sample was characterized by X-ray powder diffractogram to give Vortioxetine benzoate Form BZ2 (FIG. 4).

### Example 6: Preparation of Form BZ3 of Vortioxetine Benzoate

A solution of Vortioxetine free base in methanol (500 μL, 0.125 M, 1 eq) was mixed with a solution of benzoic acid in methanol (500 μL, 0.14M, 1.1 eq). The solvent was concentrated overnight under reduced pressure at 30° C. to give a precipitation. The precipitate was slurried in isopropanol (500 μL) at RT overnight. The solid was filtered and dried at 40° C. in a vacuum oven overnight. The sample was characterized by X-ray powder diffractogram to give Vortioxetine benzoate Form BZ3 (FIG. 5).

### Example 7: Preparation of Form IB2 of Vortioxetine Iso-Butyrate

Vortioxetine free base (15 g, 1 eq) and MIBK (150 ml, 10 vol) were charged in 250 ml reactor. The stirred mixture was heated to 76° C. and iso-butyric acid (3.6 ml, 1.1 eq) was added. The obtained clear solution was cooled to −5° C. during 1 hr and stirred for 80 hr. The solid was filtered and dried in vacuum oven at 40° C. overnight. The sample was characterized by X-ray powder diffractogram to give Vortioxetine iso-butyrate Form IB2 (FIG. 6).

### Example 8: Preparation of Form IB4 of Vortioxetine Iso-Butyrate

To a mixture of Vortioxetine free base (5 g, 1 eq) and MIBK (50 ml, 10 vol) iso-butyric acid (3.6 ml, 1.1 eq) was added. The obtained light slurry mixture was cooled down using an ice bath and stirred for 1 hr. The solid was filtered and dried at 40° C. in a vacuum oven overnight. The sample was characterized by X-ray powder diffractogram to give Vortioxetine iso-butyrate Form IB4 as (FIG. 7).

### Example 9: Preparation of Form P1 of Vortioxetine Propionate

A solution of Vortioxetine free base in methanol (500 μL, 0.125 M, 1 eq) was mixed with a solution of propionic acid in methanol (500 μL, 0.14M, 1.1 eq). The solvent was concentrated overnight under reduced pressure at 30° C. to give a precipitation. The precipitate was slurried in acetonitrile (500 μL) at RT overnight. The solid was filtered and dried at 40° C. in a vacuum oven overnight. The sample was characterized by X-ray powder diffractogram to give Vortioxetine propionate Form P1 as depicted in FIG. 8.

### Example 10: Preparation of Form P3 of Vortioxetine Propionate

A slurry mixture of Vortioxetine free base (10 g, leq) and EtOAc (50 mL, 5 vol) was heated to 40° C. and propionic acid was added (2.6 mL, 1.1 eq). The clear solution was evaporated and the residue was characterized by X-ray powder diffractogram to give Vortioxetine propionate Form P3 (FIG. 9).

### Example 11: Preparation of Form P4 of Vortioxetine Propionate

200 mg of Vortioxetine Propionate form P3 were placed in a humidity cell set to 100% RH, at RT, for 7 days. The sample was characterized by XRPD to give Vortioxetine propionate Form P4 (FIG. 10).

### Example 12: Preparation of Form IB5 of Vortioxetine Iso-Butyrate

Approximately 200 mg of Vortioxetine Isobutyrate form IB2 were manually ground in a mortar with a pestle, with several drops of water. The obtained powder was characterized by XRPD to give Vortioxetine iso-butyrate form IB35 (FIG. 11).

### Example 13: Preparation of Form BZ4 of Vortioxetine Benzoate

Vortioxetine benzoate form BZ1 (2 gr) was dissolved in DCM (15 vol) at RT. The solution was then sprayed in a spray drier at addition rate of 4 gr/min at 51° C. (outlet temperature) to give 1 g of dry Vortioxetine benzoate Form BZ4, as confirmed by XRPD.

### Example 14: Preparation of Form BZ4 of Vortioxetine Benzoate

Vortioxetine benzoate form BZ1 (2 gr) was dissolved in DCM (15 vol) at RT. The solution was then sprayed in a spray drier at addition rate of 6 gr/min at 47° C. (outlet temperature) to give 1 g of dry Vortioxetine benzoate Form BZ4 (FIG. 12).

### Example 15: Preparation of Form P1 of Vortioxetine Propionate

To a mixture of Vortioxetine free base (50 g, 1 eq) and MTBE (5 vol), water (0.1 vol) and Propionic acid (14.9 g, 1.2 eq) were added. The obtained slurry mixture was stirred and heated to 50° C. to obtain full dissolution. The solution was then cooled to about 35° C. and seeded with Vortioxetine Propionate crystals Form P3. The mixture was stirred for about 30 minutes and then cooled down during 2 hr to 0° C. The mixture was stirred at 0° C. for 2 hr and then the solid was filtered and washed with MTBE (2 vol) to give 102 g of wet material. The solid was dried under vacuum at 25° C. to give 57.6 g of Form P1 of Vortioxetine propionate as was confirmed by XRPD.

### Example 16: Preparation of Form P3 of Vortioxetine Propionate

To a mixture of Vortioxetine free base (10 g, 1 eq) and acetonitrile (8 vol), Propionic acid (2.7 g, 1.1 eq) was added. The obtained slurry mixture was heated with to 55° C. to obtain full dissolution. The mixture was filtered to remove any insoluble particles. The filtrate was then cooled to about 30° C. and seeded with Vortioxetine Propionate crystals Form P1. The mixture was stirred for about 30 minutes and then cooled down during 2 hr to (−10)° C. The mixture was stirred at (−10)° C. for 1.5 hr and then the solid was filtered and washed with acetonitrile (1 vol) to give 8.2 g of wet material. The wet material was dried under vacuum at 50° C. to give 7.1 g of Form P3 of Vortioxetine propionate as was confirmed by XRPD.

### Example 17: Preparation of Form P4 of Vortioxetine Propionate

To a mixture of Vortioxetine free base (20 g, 1 eq) and MTBE (15 vol), water (0.1 vol) and Propionic acid (6.5 g, 1.3 eq) were added. The obtained slurry mixture was stirred and heated to 50° C. to obtain full dissolution. The solution was then cooled down to about 30° C. and seeded with Vortioxetine Propionate crystals Form P1. The mixture was stirred for about 30 minutes and then cooled down during 2 hr to 0° C. The mixture was stirred at 0° C. for 19 hr and then the solid was filtered and washed with MTBE (2 vol) to give 48.4 g of wet material. The solid was dried under vacuum at 25° C. to give 24 g of Form P4 of Vortioxetine propionate.

### Example 18: Preparation of Form BZ5 of Vortioxetine Benzoate

Vortioxetine free base (20 g, 1 eq), MIBK (200 ml, 10 vol) and Benzoic acid (9.8 g, 1.2 eq) were added to a stirred reactor at 25° C. The mixture was stirred for about 24h and then the solids were filtered to give 31.9 g of wet Vortioxetine Benzoate Form BZ5 (FIG. 13).

### Example 19: Preparation of Form BZ1 of Vortioxetine Benzoate

Vortioxetine free base (20 g, 1 eq), MTBE (400 ml, 20 vol), water (4 ml, 0.2 v) and Benzoic acid (9.8 g, 1.2 eq) were added to a stirred reactor and heated to 60° C. to obtain full dissolution. The mixture was then cooled to 0° C., stirred for about 3h and then the solids were filtered. The wet solids were dried overnight under vacuum at 40° C. to give 27.4 g of dry Vortioxetine Benzoate BZ1 crystals, as was confirmed by XRPD.

### Example 20: Preparation of Form BZ1 of Vortioxetine Benzoate

Vortioxetine free base (20 g, 1 eq) and Toluene (200 ml, 10 vol), were added to a stirred reactor and heated to 70° C. to obtain full dissolution. The mixture was then filtrated to remove undissolved matter. The clear mixture was then cooled to 55° C. and Benzoic acid (9.8 g, 1.2 eq) was added. The mixture was cooled to 30° C. and seeded with crystals of Vortioxetine benzoate forms BZ1 and BZ2 The mixture was then cooled to 0° C. during 3 hours and stirred overnight, and then the solids were filtered and washed with toluene (20 ml, 1 vol). The wet solids were dried overnight under vacuum at 40° C. to give 12.4 g of dry Vortioxetine Benzoate BZ1 crystals, as was confirmed by XRPD.

### Example 21: Preparation of Form BZ1 of Vortioxetine Benzoate

Vortioxetine free base (20 g, 1 eq), n-BuOH (200 ml, 10 vol) and benzoic acid (9 g, 1.1 eq) were added to a stirred reactor and heated to 40° C. to obtain full dissolution. In a second stirred reactor, n-Heptane (300 ml, 15 vol) were charged and cooled to 0° C. Crystals of Vortioxetine benzoate form BZ2 were added to the cooled solvent. The n-BuOH mixture was then added dropwise to the cooled n-Heptane, then the mixture was stirred overnight. The solids were filtered and the wet solids were dried overnight under vacuum at 25° C. to give 17.3 g of dry Vortioxetine Benzoate BZ1 crystals, as was confirmed by XRPD.

### Example 22: Preparation of Form BZ6 of Vortioxetine Benzoate

Vortioxetine free base (20 g, 1 eq), toluene (200 ml, 10 vol) and benzoic acid (8.6 g, 1.05 eq) were added to the reactor at 25° C. The mixture was stirred and heated to 60° C. to obtain full dissolution, then cooled to 40° C. and seeded at this temperature with Vortioxetine Benzoate crystals form BZ2. The mixture was stirred for 30 min and then n-Heptane (200 ml, 10 vol) was added dropwise. The mixture was cooled to 10° C. during 2 hr and then stirred at this temperature overnight. The solid was filtered and washed with n-Heptane (20 ml, 1 vol) to give 33 g of wet Vortioxetine Benzoate salt form BZ6 (FIG. 14).

### Example 23: Preparation of Form BZ6 of Vortioxetine Benzoate

Vortioxetine free base (20 g, 1 eq), Toluene (100 ml, 5 vol) and Benzoic acid (12.9 g, 1.05 eq) were added to the reactor at 25° C. The mixture was stirred and heated to 70° C. to obtain full dissolution. The mixture was then cooled to 0° C. during 4 hr and then n-Heptane (200 ml, 10 vol) was added dropwise. The mixture was stirred at this temperature overnight. The solid was filtered and washed with n-Heptane (20 ml, 1 vol) to give 32 g of wet Vortioxetine Benzoate salt form BZ6 as was confirmed by XRPD.

### Example 24: Preparation of Form BZ6 of Vortioxetine Benzoate

Vortioxetine free base (30 g, 1 eq), Toluene (300 ml, 10 vol) and benzoic acid (8.6 g, 1.05 eq) were added to the reactor at 25° C. The mixture was stirred and heated to 60° C. to obtain full dissolution. Toluene was evaporated under vacuum until around 5 vol (around 150 ml) were remained in the reactor. The mixture was then cooled to 0° C. during 4 hr and then stirred at this temperature overnight. The solid was filtered and washed with Toluene (60 ml, 2 vol) to give 47.5 g of wet Vortioxetine Benzoate salt form BZ6, as was confirmed by XRPD.

### Example 25: Preparation of Form BZ6 of Vortioxetine Benzoate

Vortioxetine free base (130 g, 1 eq), Toluene (1950 ml, 15 vol) and Benzoic acid (55.9 g, 1.05 eq) were added to the 3 L reactor at 25° C. The mixture was stirred and heated to 60° C. to obtain full dissolution. Toluene was evaporated under vacuum until around 10 vol (around 1300 ml) were remained in the reactor. The mixture was then cooled to 38° C. and seeded with Vortioxetine benzoate crystals form BZ2. The mixture was stirred at this temperature for about 2 hr and then n-Heptane (1300 ml, 10 vol) was added dropwise. The mixture was then cooled to 0° C. during 4 hr and stirred at this temperature overnight. The solid was filtered and washed with Toluene (60 ml, 2 vol) to give 221.1 g of wet Vortioxetine Benzoate salt form BZ6, as was confirmed by XRPD.

### Example 26: Preparation of Form BZ7 of Vortioxetine Benzoate

Vortioxetine free base (30 g, 1 eq), Toluene (450 ml, 15 vol) and benzoic acid (13.5 g, 1.1 eq) were added to the reactor at 25° C. The mixture was stirred and heated to 60° C. to obtain full dissolution. Toluene was evaporated under vacuum until around 10 vol (around 300 ml) were remained in the reactor. The mixture was then cooled to 0° C. during 4 hr and then stirred at this temperature overnight. The solid was filtered and washed with Toluene (60 ml, 2 vol) to give 51.4 g of wet Vortioxetine Benzoate salt. The salt was then dried at 90° C. under vacuum overnight to give 39.3 g of dry Vortioxetine Benzoate salt form BZ7 (FIG. 15).

### Example 27: Preparation of Form BZ8 of Vortioxetine Benzoate

A starting mixture of benzoic acid with Vortioxetine benzoate salt Form BZ2 was prepared (5% (w/w acid in BZ2). A vial was charged with about 60 mg of the mixture, and suspended in 3 ml solution of toluene:heptane (1:1) at RT with a mechanical stirrer at 700 rpm.

The suspension was fully dissolved while heating to 90° C., held for 10 min, then cooled to 0° C. at a rate of 0.5° C./min and stirred for about 16 hours. The precipitated solid was filtered and tested by XRPD-Vortioxetine benzoate salt form BZ8 (FIG. 16).

### Example 28: Preparation of Form BZ2 of Vortioxetine Benzoate

Vortioxetine free base (1 eq), Toluene (15 vol) and benzoic acid (1.05 eq) were added to a stirred reactor at room temperature. The mixture was heated to 60-70° C. to obtain full dissolution. The solution was distillated to 10 vol of the solvent level followed by filtration Heptane (5 vol) was added while keeping the temperature mixture at 55-65° C. and stirred for additional hour. Vortioxetine benzoate form BZ2 seeds (0.5 w/w) were added and stirred for additional 4 hours. The mixture was then cooled during 4 hours to 15° C. and stirred for additional 24 hours. The solid was filtered, washed with heptane (2*2 vol) and the wet solid was dried under vacuum with gradual heating from 40° C. to 110° C. for about 4 days to give Vortioxetine benzoate form BZ2.

Further aspects and embodiments of the present invention are set out in the following numbered clauses:

1. A Vortioxetine salt selected from: Vortioxetine butyrate, Vortioxetine iso-butyrate, Vortioxetine benzoate and Vortioxetine propionate.

2. Vortioxetine butyrate designated as Form B1 characterized by data selected from one or more of the following: an XRPD pattern having peaks at 6.7, 9.6, 15.5, 16.0 and 27.2 degrees 2-theta±0.2 degrees 2-theta; an XRPD pattern substantially as depicted in FIG. 1; or combinations of these data.

3. Vortioxetine butyrate designated as Form B2 characterized by data selected from one or more of the following: an XRPD pattern having peaks at 7.6, 10.1, 11.8, 15.2 and 18.1 degrees two theta±0.2 degrees two theta; an XRPD pattern substantially as depicted in FIG. 2; or combinations of these data.

4. Vortioxetine benzoate designated as Form BZ1 characterized by data selected from one or more of the following: an XRPD pattern having peaks at 6.7, 9.5, 10.4, 14.7 and 16.9 degrees two theta±0.2 degrees two theta; an XRPD pattern substantially as depicted in FIG. 3; or combinations of these data.

5. Vortioxetine benzoate designated as Form BZ2 characterized by data selected from one or more of the following: an XRPD pattern having peaks at 6.3, 7.1, 9.0, 11.5 and 21.7 degrees two theta±0.2 degrees two theta; an XRPD pattern substantially as depicted in FIG. 4; or combinations of these data.

6. Vortioxetine benzoate designated as Form BZ3 characterized by data selected from one or more of the following: an XRPD pattern having peaks at 7.0, 10.1, 14.0, 15.7 and 18.5 degrees two theta±0.2 degrees two theta; an XRPD pattern substantially as depicted in FIG. 5; or combinations of these data.

7. Vortioxetine iso butyrate designated as Form IB32 characterized by data selected from one or more of the following: an XRPD pattern having peaks at 7.1, 8.7, 10.3, 12.0, and 14.8 degrees two theta±0.2 degrees two theta; an XRPD pattern substantially as depicted in FIG. 6; or combinations of these data.

8. Vortioxetine iso butyrate designated as Form IB34 characterized by data selected from one or more of the following: an XRPD pattern having peaks at 6.6, 15.7, 18.7, 20.2 and 23.5 degrees two theta±0.2 degrees two theta; an XRPD pattern substantially as depicted in FIG. 7; or combinations of these data.

9. Vortioxetine propionate designated as Form P1 characterized by data selected from one or more of the following: an XRPD pattern having peaks at 8.6, 16.1, 18.2, 21.3 and 22.1 degrees two theta±0.2 degrees two theta; an XRPD pattern substantially as depicted in FIG. 8; or combinations of these data.

10. Vortioxetine propionate designated as Form P3 characterized by data selected from one or more of the following: an XRPD pattern having peaks at 7.2, 8.0, 9.8, 11.6 and 13.6 degrees two theta±0.2 degrees two theta; an XRPD pattern substantially as depicted in FIG. 9; or combinations of these data.

11. Vortioxetine propionate designated as Form P4, characterized by data selected from one or more of the following: an XRPD pattern having peaks at 6.6, 11.1, 20.6, 29.7 and 33.7 degrees two theta±0.2 degrees two theta; an XRPD pattern substantially as depicted in FIG. 10; or combinations of these data.

12. Vortioxetine iso butyrate designated as Form IB35, characterized by data selected from one or more of the following: an XRPD pattern having peaks at 10.4, 11.8, 14.0, 20.2 and 26.0 degrees two theta±0.2 degrees two theta; an XRPD pattern substantially as depicted in FIG. 11; or combinations of these data.

13. Vortioxetine benzoate designated as Form BZ4, characterized by data selected from one or more of the following: an XRPD pattern having peaks at 8.4, 9.8, 14.9, 18.9 and 22.4 degrees two theta±0.2 degrees two theta; an XRPD pattern substantially as depicted in FIG. 12; or combinations of these data.

14. Vortioxetine benzoate designated as Form BZ5, characterized by data selected from one or more of the following: an XRPD pattern having peaks at 6.6, 8.6, 11.5, 12.0 and 23.1 degrees two theta±0.2 degrees two theta; an XRPD pattern substantially as depicted in FIG. 13; or combinations of these data.

15. Vortioxetine benzoate designated as Form BZ6, characterized by data selected from one or more of the following: an XRPD pattern having peaks at 8.1, 8.5, 17.2, 19.3, and 20.0 degrees two theta±0.2 degrees two theta; an XRPD pattern substantially as depicted in FIG. 14; or combinations of these data.

16. Vortioxetine benzoate designated as Form BZ7, characterized by data selected from one or more of the following: an XRPD pattern having peaks at 9.9, 13.2, 16.0, 20.9 and 26.0 degrees two theta±0.2 degrees two theta; an XRPD pattern substantially as depicted in FIG. 15; or combinations of these data.

17. Vortioxetine benzoate designated as Form BZ8, characterized by data selected from one or more of the following: an XRPD pattern having peaks at 7.1, 8.1, 15.6, 17.3 and 20.1 degrees two theta±0.2 degrees two theta; an XRPD pattern substantially as depicted in FIG. 16; or combinations of these data.

18. Methods of treating major depressive disorder (MDD), comprising administering a therapeutically effective amount of any one of the salts and solid state forms of Vortioxetine salts of the present disclosure or combinations thereof, or at least one of the above pharmaceutical compositions or formulations, to a subject suffering from major depressive disorder (MDD), or otherwise in need of the treatment.

1. A crystalline form of Vortioxetine benzoate designated as BZ2, wherein said crystalline form BZ2 is characterized by data selected from one or more of the following: an XRPD pattern having peaks at 6.3, 7.1, 9.0, 11.5 and 21.7 degrees two theta±0.2 degrees two theta; an XRPD pattern substantially as depicted in FIG. 4; or combinations of these data.

2. The crystalline form BZ2 of Vortioxetine benzoate according to claim 1, wherein said crystalline form BZ2 is characterized by an XRPD pattern having peaks at 6.3, 7.1, 9.0, 11.5 and 21.7 degrees two theta±0.2 degrees two theta; wherein said crystalline form BZ2 is further characterized by an XRPD pattern having one, two, three, four or five additional peaks selected from the group consisting of 13.3, 14.2, 16.7, 18.1 and 22.6 degrees two theta±0.2 degrees two theta.

3. The crystalline form BZ2 of Vortioxetine benzoate according to claim 1, wherein said crystalline form BZ2 is anhydrous.

4. A process for preparing the crystalline form BZ2 of Vortioxetine benzoate according to claim 1, wherein said process comprises the steps of: obtaining a reaction mixture of a Vortioxetine base and benzoic acid and then precipitating Vortioxetine benzoate in said crystalline form BZ2 from said reaction mixture.

5. The process of claim 4, wherein the reaction mixture comprises an organic solvent.

6. The process according to claim 5, wherein said organic solvent is selected from ethyl acetate and heptane.

7. A pharmaceutical composition comprising the crystalline form BZ2 of Vortioxetine benzoate according to claim 1 as active ingredient.

8. A pharmaceutical formulation comprising the crystalline form BZ2 of Vortioxetine benzoate according to claim 1 and at least one pharmaceutically acceptable excipient.

9. A process for preparing the pharmaceutical formulation according to claim 8, wherein said process comprises the step of combining the crystalline form BZ2 of Vortioxetine benzoate according to claim 1 with at least one pharmaceutically acceptable excipient.

10. A method of treating a major depressive disorder (MDD), wherein said method comprises the step of administering to a subject suffering from a major depressive disorder (MDD), or otherwise in need of such treatment, a therapeutically effective amount of a medicament selected from the group consisting of: a pharmaceutical formulation of a Vortioxetine benzoate salt; the crystalline form BZ2 of Vortioxetine benzoate according to claim 1; and a pharmaceutical composition comprising the crystalline form BZ2 of Vortioxetine benzoate as active ingredient.
